SEQUENCE LISTING (15 sequences)

SEQ ID NO: 1 - protein, 15 aa, Homo sapiens
KFGADARALMLQGVD

SEQ ID NO: 2 - protein, 15 aa, Homo sapiens
LMLQGVDLLADAVAV

SEQ ID NO: 3 - protein, 15 aa, Homo sapiens
EKKDRVTDALNATRA

SEQ ID NO: 4 - protein, 15 aa, Homo sapiens
IQSIVPALEIANAHR

SEQ ID NO: 5 - protein, 11 aa, Homo sapiens
AELKKQSKPVT

SEQ ID NO: 6 - protein, 15 aa, Homo sapiens
EVVEGMQFDRGYLSP

SEQ ID NO: 7 - protein, 15 aa, Homo sapiens
KEEKDPGMGAMGGMG

SEQ ID NO: 8 - protein, 15 aa, Homo sapiens
VTDALNATRAAVEEG

SEQ ID NO: 9 - protein, 17 aa, Homo sapiens
TLVLNRLKVGLQVVAVK

SEQ ID NO: 10 - protein, 13 aa, Homo sapiens
EEIAQVATISANG

SEQ ID NO: 11 - protein, 13 aa, Homo sapiens
AVKAPGHFDNRKN

SEQ ID NO: 12 - protein, 12 aa, Homo sapiens
KKQSKPVTTPEE

SEQ ID NO: 13 - protein, 15 aa, Homo sapiens
EIPKEEKDPGMGAMG

SEQ ID NO: 14 - protein, 300 aa, Homo sapiens
MLRLPTVFRQMRPVSRVLAPHLTRAYAKDVKFGADARALMLQGVDLLADAVAVTMGPKGRTVIIEQSWGSPKVTKDGVTVAKSIDLKDKYKNIGAKLVQDVANNTNEEAGDGTTTATVLARSIAKEGFEKISKGANPVEIRRGVMLAVDAVIAELKKQSKPVTTPEEIAQVATISANGDKEIGNIISDAMKKVGRKGVITVKDGKTLNDELEIIEGMKFDRGYISPYFINTSKGQKCEFQDAYVLLSEKKISSIQSIVPALEIANAHRKPLVIIAEDVDGEALSTLVLNRLKVGLQVVAV

SEQ ID NO: 15 - protein, 15 aa, Homo sapiens
EIIEGMKFDRGYISP

(19) United States
(12) Patent Application Publication
(10) Pub. No.: US 2023/0011870 A1
(43) Pub. Date: Jan. 12, 2023

(54) NANOVACCINE FOR HEART FAILURE

(71) Applicant: THE METHODIST HOSPITAL SYSTEM, Houston, TX (US)

(72) Inventors: Guillermo TORRE-AMIONE, Houston, TX (US); Keith Adam YOUKER, Houston, TX (US); Haifa SHEN, Bellaire, TX (US)

(21) Appl. No.: 17/777,335

(22) PCT Filed: Nov. 18, 2020
(86) PCT No.: PCT/US2020/061017

§ 371 Date: May 17, 2022

Related U.S. Application Data
(60) Provisional application No. 62/936,876, filed on Nov. 18, 2019.

(51) Int. Cl. A61K 39/00; A61P 9/04

(52) U.S. Cl. CPC A61K 39/0005; A61P 9/04; A61K 2039/55505

(57) ABSTRACT

Disclosed are compositions and methods for treating heart failure and cardiac damage that can lead to heart failure if left untreated comprising administering to a subject in need thereof a composition comprising an HSP60 derived peptide and an adjuvant.

This application claims the benefit of U.S. Provisional Application No. 62/936,876, filed on Nov. 18, 2019, which is incorporated herein by reference in its entirety.

### I. BACKGROUND

1. Currently heart failure has a great impact on morbidity and mortality globally and it is estimated to affect at least 26 million people worldwide. Current therapies are focused on the Renin-Angiotensin-Aldosterone-System (RAAS) or directly on cardiac contractility but despite advances, the mortality/morbidity remains high and affected patients have a poor quality of life. Currently the only definitive cure is cardiac transplantation which is currently limited to around 50,000 heart transplants in the US each year due to the low availability of donor organs. What are needed are new non-invasive treatments for heart failure and other debilitating cardiac injuries.

### II. SUMMARY

2. Disclosed are methods and compositions related to HSP60 peptide vaccines.

3. In one aspect, disclosed herein are compositions comprising a HSP60 peptide (such as, for example, KFGADARALMLQGVD (SEQ ID NO: 1), LMLQGVDLLADAVAV (SEQ ID NO: 2), EKKDRVTDALNATRA (SEQ ID NO: 3), IQSIVPALEIANAHR (SEQ ID NO: 4), AELKKQSKPVT (SEQ ID NO: 5), EVVEGMQFDRGYLSP (SEQ ID NO: 6), KEEKDPGMGAMGGMG (SEQ ID NO: 7), VTDALNATRAAVEEG (SEQ ID NO: 8), TLVLNRLKVGLQVVAVK (SEQ ID NO: 9), EEIAQVATISANG (SEQ ID NO: 10), AVKAPGHFDNRKN (SEQ ID NO: 11), KKQSKPVTTPEE (SEQ ID NO: 12), EIPKEEKDPGMGAMG (SEQ ID NO: 13), or EIIEGMKFDRGYISP (SEQ ID NO: 15)) and an adjuvant (such as, for example, alum, aluminum hydroxide, aluminum phosphate, mixed aluminum salts, a nonionic block co-polymer (such as, for example CRL-1005), Adjuvant system 04 (AS04), or Adjuvant system 03 (AS03)). For example, disclosed herein are composition comprising an HSP60 peptide (such as, for example, KFGADARALMLQGVD (SEQ ID NO: 1), LMLQGVDLLADAVAV (SEQ ID NO: 2), EKKDRVTDALNATRA (SEQ ID NO: 3), IQSIVPALEIANAHR (SEQ ID NO: 4), AELKKQSKPVT (SEQ ID NO: 5), EVVEGMQFDRGYLSP (SEQ ID NO: 6), KEEKDPGMGAMGGMG (SEQ ID NO: 7), VTDALNATRAAVEEG (SEQ ID NO: 8), TLVLNRLKVGLQVVAVK (SEQ ID NO: 9), EEIAQVATISANG (SEQ ID NO: 10), AVKAPGHFDNRKN (SEQ ID NO: 11), KKQSKPVTTPEE (SEQ ID NO: 12), EIPKEEKDPGMGAMG (SEQ ID NO: 13), or EIIEGMKFDRGYISP (SEQ ID NO: 15)) and alum.

4. Also disclosed herein are compositions comprising an HSP60 peptide and an adjuvant of any preceding aspect, wherein the HSP60 peptide is conjugated to the adjuvant.

5. In one aspect, disclosed here are methods of treating, reducing, inhibiting, decreasing, ameliorating, and/or preventing heart failure, cardiac disease, or cardiac dysfunction progression to chronic heart failure in a subject comprising administering to the subject the composition of any preceding aspect. For example, in one aspect, disclosed herein are methods of treating, reducing, inhibiting, decreasing, ameliorating, and/or preventing heart failure, cardiac disease, or cardiac dysfunction progression to chronic heart failure in a subject comprising administering to the subject a composition comprising an HSP60 peptide (such as, for example, KFGADARALMLQGVD (SEQ ID NO: 1), LMLQGVDLLADAVAV (SEQ ID NO: 2), EKKDRVTDALNATRA (SEQ ID NO: 3), IQSIVPALEIANAHR (SEQ ID NO: 4), AELKKQSKPVT (SEQ ID NO: 5), EVVEGMQFDRGYLSP (SEQ ID NO: 6), KEEKDPGMGAMGGMG (SEQ ID NO: 7), VTDALNATRAAVEEG (SEQ ID NO: 8), TLVLNRLKVGLQVVAVK (SEQ ID NO: 9), EEIAQVATISANG (SEQ ID NO: 10), AVKAPGHFDNRKN (SEQ ID NO: 11), KKQSKPVTTPEE (SEQ ID NO: 12), EIPKEEKDPGMGAMG (SEQ ID NO: 13), or EIIEGMKFDRGYISP (SEQ ID NO: 15)) and an adjuvant (such as, for example, alum, aluminum hydroxide, aluminum phosphate, mixed aluminum salts, a nonionic block co-polymer (such as, for example CRL-1005), Adjuvant system 04 (AS04), or Adjuvant system 03 (AS03)).

6. In one aspect, disclosed herein are methods of treating, reducing, inhibiting, decreasing, ameliorating, and/or preventing heart failure, cardiac disease, or cardiac dysfunction progression to chronic heart failure of any of preceding aspect, wherein the cardiac disease or dysfunction comprises coronary artery disease, myocardial infarction, hypertension, cardiomyopathy, myocarditis, congenital heart defect, ischemia reperfusion injury, myocardial ischemia, myocardial reperfusion, subendocardial ischemia, Takayasu's arteritis, atrial fibrillation, hemorrhagic strokes, transient ischemia attack, or heart arrythmias.

7. Also disclosed herein are methods of treating, reducing, inhibiting, decreasing, ameliorating, and/or preventing heart failure, cardiac disease, or cardiac dysfunction progression to chronic heart failure of any of preceding aspect, wherein the heart failure is acute heart failure.

8. In one aspect, disclosed herein are methods of treating, reducing, inhibiting, decreasing, ameliorating, and/or preventing heart failure, cardiac disease, or cardiac dysfunction progression to chronic heart failure of any of preceding aspect, wherein the heart failure is surgically induced (such as, for example, surgically induced ischemic/reperfusion events occurring during the preservation of organs for transplant or during cardiac surgery (including coronary artery bypass surgery, stent implantation, heart valve replacements, myectomy, transmyocardial revascularization, congenital heart surgery, angioplasty, atherectomy, and/or cardiomyoplasty.

9. Also disclosed herein are methods of treating, reducing, inhibiting, decreasing, ameliorating, and/or preventing heart failure, cardiac disease, or cardiac dysfunction progression to chronic heart failure of any of preceding aspect, wherein the composition is administered to the subject prior to the onset of heart failure, wherein the heart failure is acute heart failure and the composition is administered to the subject after the onset of symptoms associated with heart failure. In some instances, wherein the heart failure is surgically induced, the composition comprising an HSP60 peptide and an adjuvant is administered before, during, or after surgery.

10. In one aspect, disclosed here are methods of treating, reducing, inhibiting, decreasing, ameliorating, and/or preventing heart failure, cardiac disease, or cardiac dysfunction in a subject comprising administering to the subject the composition of any preceding aspect. For example, in one aspect, disclosed herein are methods of treating, reducing, inhibiting, decreasing, ameliorating, and/or preventing heart failure, cardiac disease, or cardiac dysfunction in a subject comprising administering to the subject a composition comprising an HSP60 peptide (such as, for example, KFGADARALMLQGVD (SEQ ID NO: 1), LMLQGVDLLADAVAV (SEQ ID NO: 2), EKKDRVTDALNATRA (SEQ ID NO: 3), IQSIVPALEIANAHR (SEQ ID NO: 4), AELKKQSKPVT (SEQ ID NO: 5), EVVEGMQFDRGYLSP (SEQ ID NO: 6), KEEKDPGMGAMGGMG (SEQ ID NO: 7), VTDALNATRAAVEEG (SEQ ID NO: 8), TLVLNRLKVGLQVVAVK (SEQ ID NO: 9), EEIAQVATISANG (SEQ ID NO: 10), AVKAPGHFDNRKN (SEQ ID NO: 11), KKQSKPVTTPEE (SEQ ID NO: 12), EIPKEEKDPGMGAMG (SEQ ID NO: 13), or EIIEGMKFDRGYISP (SEQ ID NO: 15)) and an adjuvant (such as, for example, alum, aluminum hydroxide, aluminum phosphate, mixed aluminum salts, a nonionic block co-polymer (such as, for example CRL-1005), Adjuvant system 04 (AS04), or Adjuvant system 03 (AS03)).

11. In one aspect, disclosed herein are methods of treating, reducing, inhibiting, decreasing, ameliorating, and/or preventing heart failure, cardiac disease, or cardiac dysfunction of any of preceding aspect, wherein the cardiac disease or dysfunction comprises coronary artery disease, myocardial infarction, hypertension, cardiomyopathy, myocarditis, congenital heart defect, ischemia reperfusion injury, myocardial ischemia, myocardial reperfusion, subendocardial ischemia, Takayasu's arteritis, atrial fibrillation, hemorrhagic strokes, transient ischemia attack, or heart arrythmias.

12. Also disclosed herein are methods of treating, reducing, inhibiting, decreasing, ameliorating, and/or preventing heart failure, cardiac disease, or cardiac dysfunction of any of preceding aspect, wherein the heart failure is acute heart failure or chronic heart failure.

13. In one aspect, disclosed herein are methods of treating, reducing, inhibiting, decreasing, ameliorating, and/or preventing heart failure, cardiac disease, or cardiac dysfunction of any of preceding aspect, wherein the heart failure is surgically induced (such as, for example, surgically induced ischemic/reperfusion events occurring during the preservation of organs for transplant or during cardiac surgery (including coronary artery bypass surgery, stent implantation, heart valve replacements, myectomy, transmyocardial revascularization, congenital heart surgery, angioplasty, atherectomy, and/or cardiomyoplasty.

14. Also disclosed herein are methods of treating, reducing, inhibiting, decreasing, ameliorating, and/or preventing heart failure, cardiac disease, or cardiac dysfunction of any of preceding aspect, wherein the composition is administered to the subject prior to the onset of heart failure, wherein the heart failure is a chronic heart failure and the composition is administered to the subject after the onset of heart failure, or wherein the heart failure is acute heart failure and the composition is administered to the subject after the onset of symptoms associated with heart failure. In some instances, wherein the heart failure is surgically induced, the composition comprising an HSP60 peptide and an adjuvant is administered before, during, or after surgery.

15.

### III. BRIEF DESCRIPTION OF THE DRAWINGS

16. The accompanying drawings, which are incorporated in and constitute a part of this specification, illustrate several embodiments and together with the description illustrate the disclosed compositions and methods.

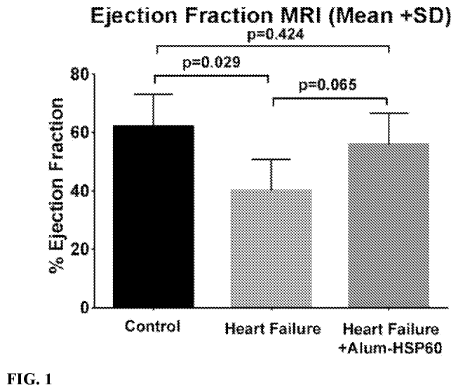

17. FIG. 1 shows ejection fraction of the heart with or without HSP60 peptide+Alum therapy.

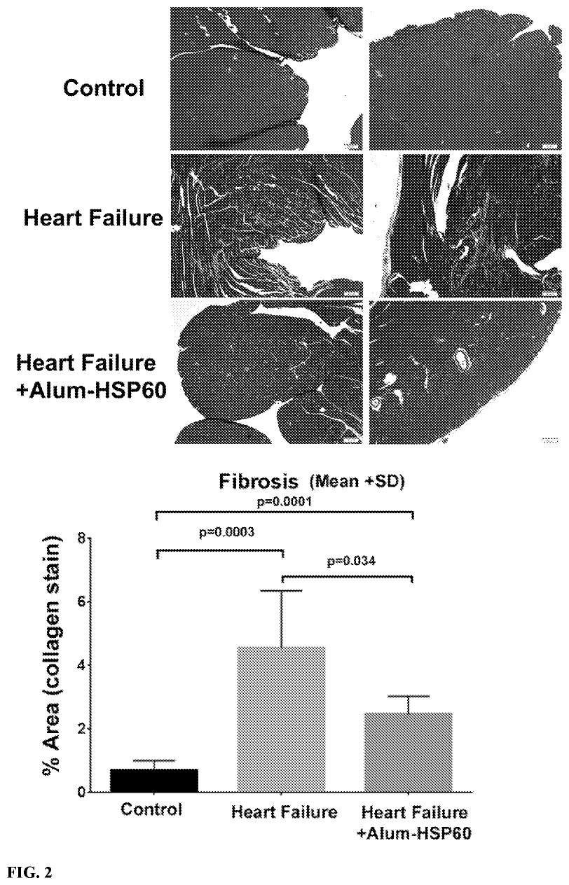

18. FIG. 2 shows fibrosis staining in treated (HSP60 peptide+Alum) and untreated tissue.

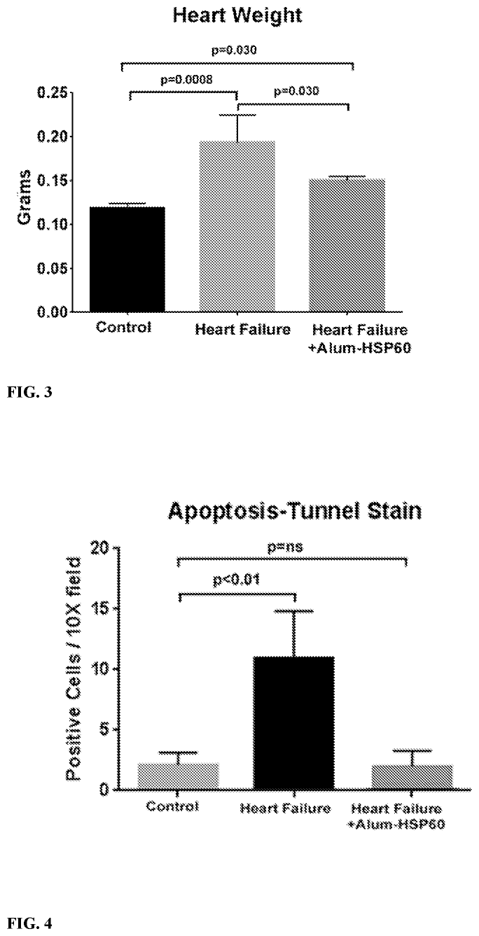

19. FIG. 3 shows the quantitation of fibrosis in treated (HSP60 peptide+Alum) and untreated tissue.

20. FIG. 4 shows a comparison apoptosis as measured by tunnel stain between treated (HSP60 peptide+Alum) and untreated tissue.

### IV. DETAILED DESCRIPTION

21. Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

22. In this specification and in the claims which follow, reference will be made to a number of terms which shall be defined to have the following meanings:

23. As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

24. Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

25. “Optional” or “optionally” means that the subsequently described event or circumstance may or may not occur, and that the description includes instances where said event or circumstance occurs and instances where it does not.

26. A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

27. “Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

28. By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

29. “Treat,” “treating,” “treatment,” and grammatical variations thereof as used herein, include the administration of a composition with the intent or purpose of partially or completely preventing, delaying, curing, healing, alleviating, relieving, altering, remedying, ameliorating, improving, stabilizing, mitigating, and/or reducing the intensity or frequency of one or more a diseases or conditions, a symptom of a disease or condition, or an underlying cause of a disease or condition. Treatments according to the invention may be applied preventively, prophylactically, pallatively or remedially. Prophylactic treatments are administered to a subject prior to onset (e.g., before obvious signs of cancer), during early onset (e.g., upon initial signs and symptoms of cancer), or after an established development of cancer. Prophylactic administration can occur for day(s) to years prior to the manifestation of symptoms of an infection.

30. By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

31. “Biocompatible” generally refers to a material and any metabolites or degradation products thereof that are generally non-toxic to the recipient and do not cause significant adverse effects to the subject.

32. “Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

33. A “control” is an alternative subject or sample used in an experiment for comparison purposes. A control can be “positive” or “negative.”

34. The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

35. “Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

36. A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

37. “Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

38. “Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

39. “Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

40. “Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

41. The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

42. Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. COMPOSITIONS

43. Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular HSP60 peptide is disclosed and discussed and a number of modifications that can be made to a number of molecules including the HSP60 peptide are discussed, specifically contemplated is each and every combination and permutation of HSP60 peptide and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

44. It is shown herein that HSP60 doubled in end-stage heart failure. In heart failure, HSP60 is in the plasma membrane fraction, on the cell surface, and in the plasma. Membrane HSP60 correlates with increased apoptosis. Release of HSP60 can also activate the innate immune system, promoting a proinflammatory state, including an increase in TNF-alpha. Thus, abnormal trafficking of HSP60 to the cell surface can be an early trigger for myocyte loss and the progression of heart failure. Additionally, HSP60 plays a role in apoptosis of cells. By immunizing a subject undergoing heart failure or at risk for heart failure with HSP60 peptides, the peptides can not only competitively inhibit full-length HSP60 protein, but can be used to generate anti-HSP60 antibodies, which can inhibit apoptosis and bind HSP60 at the site of cardiac dysfunction, cardiac disease, and/or heart failure. In one aspect, disclosed herein are compositions comprising a HSP60 peptide (such as, for example, KFGADARALMLQGVD (SEQ ID NO: 1), LMLQGVDLLADAVAV (SEQ ID NO: 2), EKKDRVTDALNATRA (SEQ ID NO: 3), IQSIVPALEIANAHR (SEQ ID NO: 4), AELKKQSKPVT (SEQ ID NO: 5), EVVEGMQFDRGYLSP (SEQ ID NO: 6), KEEKDPGMGAMGGMG (SEQ ID NO: 7), VTDALNATRAAVEEG (SEQ ID NO: 8), TLVLNRLKVGLQVVAVK (SEQ ID NO: 9), EEIAQVATISANG (SEQ ID NO: 10), AVKAPGHFDNRKN (SEQ ID NO: 11), KKQSKPVTTPEE (SEQ ID NO: 12), EIPKEEKDPGMGAMG (SEQ ID NO: 13), or EIIEGMKFDRGYISP (SEQ ID NO: 15)). In some aspects, the composition comprises a therapeutically effective amount of a HSP60 peptide.

45. It is understood and herein contemplated that the disclosed composition comprise a peptide vaccine that will induce an immune response to HSP60. The disclosed peptide comprise designed epitopes are the antigenic determinants within the larger HSP60 protein, these peptides are considered sufficient for activation of the appropriate cellular and humoral responses, while eliminating any potential allergenic and/or reactogenic responses. Additionally, the HSP60 peptide vaccines disclosed herein can be used for induction of broad-spectrum immunity against multiple serological variants by formulating multiple non-contiguous immunodominant epitopes and/or epitopes conserved between different serovars. However, due to the relatively small size of peptides, the HSP60 peptides alone are often weakly immunogenic by themselves and therefore require carrier molecules, to add chemical stability and adjuvanting, for the induction of a robust immune response.

46. Accordingly, in one aspect, disclosed herein are compositions comprising a HSP60 peptide an adjuvant. As used herein adjuvants can comprise any substance that can enhance an immune response to a weakly immunogenic antigen. Examples of adjuvants that can be used for human use and in combination with the disclosed HSP60 peptides include, but are not limited to alum, aluminum hydroxide, aluminum phosphate, mixed aluminum salts, a nonionic block co-polymer (such as, for example CRL-1005), Adjuvant system 04 (AS04)(which is a combination of aluminum hydroxide and monophosphoryl lipid A (MPL)), or Adjuvant system 03 (AS03) (which is made up of the oily compounds, D,L-alpha-tocopherol (vitamin E) and squalene, and an emulsifier, polysorbate 80). Thus, in one aspect, disclosed herein are compositions comprising a HSP60 peptide (such as, for example, KFGADARALMLQGVD (SEQ ID NO: 1), LMLQGVDLLADAVAV (SEQ ID NO: 2), EKKDRVTDALNATRA (SEQ ID NO: 3), IQSIVPALEIANAHR (SEQ ID NO: 4), AELKKQSKPVT (SEQ ID NO: 5), EVVEGMQFDRGYLSP (SEQ ID NO: 6), KEEKDPGMGAMGGMG (SEQ ID NO: 7), VTDALNATRAAVEEG (SEQ ID NO: 8), TLVLNRLKVGLQVVAVK (SEQ ID NO: 9), EEIAQVATISANG (SEQ ID NO: 10), AVKAPGHFDNRKN (SEQ ID NO: 11), KKQSKPVTTPEE (SEQ ID NO: 12), EIPKEEKDPGMGAMG (SEQ ID NO: 13), or EIIEGMKFDRGYISP (SEQ ID NO: 15)), including, but not limited to, therapeutically effective amount of a HSP60 peptide, and an adjuvant (such as, for example, alum, aluminum hydroxide, aluminum phosphate, mixed aluminum salts, a nonionic block co-polymer (such as, for example CRL-1005), Adjuvant system 04 (AS04), or Adjuvant system 03 (AS03)). For example, disclosed herein are composition comprising an HSP60 peptide (such as, for example, KFGADARALMLQGVD (SEQ ID NO: 1), LMLQGVDLLADAVAV (SEQ ID NO: 2), EKKDRVTDALNATRA (SEQ ID NO: 3), IQSIVPALEIANAHR (SEQ ID NO: 4), AELKKQSKPVT (SEQ ID NO: 5), EVVEGMQFDRGYLSP (SEQ ID NO: 6), KEEKDPGMGAMGGMG (SEQ ID NO: 7), VTDALNATRAAVEEG (SEQ ID NO: 8), TLVLNRLKVGLQVVAVK (SEQ ID NO: 9), EEIAQVATISANG (SEQ ID NO: 10), AVKAPGHFDNRKN (SEQ ID NO: 11), KKQSKPVTTPEE (SEQ ID NO: 12), EIPKEEKDPGMGAMG (SEQ ID NO: 13), or EIIEGMKFDRGYISP (SEQ ID NO: 15)) and alum.

47. It is understood and herein contemplated that the peptide and the adjuvant can be part of the same composition as separate components or linked together. In one aspect disclosed herein are compositions comprising any of the HSP60 peptides and an adjuvants disclosed herein, wherein the HSP60 peptide is conjugated to the adjuvant.

1. Sequence Similarities

48. It is understood that as discussed herein the use of the terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

49. In general, it is understood that one way to define any known variants and derivatives or those that might arise, of the disclosed genes and proteins herein, is through defining the variants and derivatives in terms of homology to specific known sequences. This identity of particular sequences disclosed herein is also discussed elsewhere herein. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level.

50. Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

51. It is understood that any of the methods typically can be used and that in certain instances the results of these various methods may differ, but the skilled artisan understands if identity is found with at least one of these methods, the sequences would be said to have the stated identity, and be disclosed herein.

52. For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

53. The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

54. Thus, the compositions can comprise, in addition to the disclosed HSP60 peptides and adjuvants, for example, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J. Resp. Cell. Mol. Biol. 1:95-100 (1989); Felgner et al. Proc. Natl. Acad. Sci USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

55. The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). These techniques can be used for a variety of other specific cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

56. A similar group of colloids to liposomes that has been explored for the delivery of antigens are virosomes, transfersomes, archeosomes, niosomes and cochleates. Niosomes are made of non-ionic surfactants and are considered to be more stable than conventional liposome. Virosomes are composed of assembled viral membrane protein which render them enhanced binding to APCs and promote cytosolic delivery. Structurally, virosomes comprise 70% of naturally occurring phospholipids and 30% envelop phospholipids originating from the influenza virus. Virosomal delivery of antigens to APCS is known to enhance MHC class I and MHC class II presentation and induce both B- and T-cell responses. Virosomes are excellent adjuvant systems and are biodegradable, non-toxic, and do not induce antibodies against themselves.

57. In some aspect, the HSP60 peptides disclosed herein (such as, for example, SEQ ID NO: 1, SEQ ID NO: 2, SEQ ID NO: 3 SEQ ID NO: 4 SEQ ID NO: 5 SEQ ID NO: 6 SEQ ID NO: 7 SEQ ID NO: 8, SEQ ID NO: 9, SEQ ID NO: 10, SEQ ID NO: 11, SEQ ID NO: 12, SEQ ID NO: 13, and/or SEQ ID NO: 15) with or without further adjuvant can also be delivered in a hydrogel or microencapsulated for controlled release, decrease degradation of the peptide, and to provide some adjuvant effect. The hydrogels, ananoparticles, and microcapsules comprising said HSP60 peptides disclosed herein can be made using any suitable biodegradable polymer. “Polymer” refers to a relatively high molecular weight organic compound, natural or synthetic, whose structure can be represented by a repeated small unit, the monomer. Non-limiting examples of polymers include polyethylene, rubber, cellulose. Synthetic polymers are typically formed by addition or condensation polymerization of monomers. The term “copolymer” refers to a polymer formed from two or more different repeating units (monomer residues). By way of example and without limitation, a copolymer can be an alternating copolymer, a random copolymer, a block copolymer, or a graft copolymer. It is also contemplated that, in certain aspects, various block segments of a block copolymer can themselves comprise copolymers. The term “polymer” encompasses all forms of polymers including, but not limited to, natural polymers, synthetic polymers, homopolymers, heteropolymers or copolymers, addition polymers, etc.

58. In one aspect, the hydrogel can comprise a biocompatible polymer (such as, for example, alginate). Such polymers can also serve to slowly release any of the HSP60 peptides and adjuvants disclosed herein into the tissue. As used herein biocompatible polymers include, but are not limited to polysaccharides; hydrophilic polypeptides; poly(amino acids) such as poly-L-glutamic acid (PGS), gamma-polyglutamic acid, poly-L-aspartic acid, poly-L-serine, or poly-L-lysine; polyalkylene glycols and polyalkylene oxides such as polyethylene glycol (PEG), polypropylene glycol (PPG), and poly(ethylene oxide) (PEO); poly(oxyethylated polyol); poly(olefinic alcohol); polyvinylpyrrolidone); poly(hydroxyalkylmethacrylamide); poly(hydroxyalkylmethacrylate); poly(saccharides); poly(hydroxy acids); poly(vinyl alcohol), polyhydroxyacids such as poly(lactic acid), poly (gly colic acid), and poly (lactic acid-co-glycolic acids); polyhydroxyalkanoates such as poly3-hydroxybutyrate or poly4-hydroxybutyrate; polycaprolactones; poly(orthoesters); polyanhydrides; poly(phosphazenes); poly(lactide-co-caprolactones); polycarbonates such as tyrosine polycarbonates; polyamides (including synthetic and natural polyamides), polypeptides, and poly(amino acids); polyesteramides; polyesters; poly(dioxanones); poly(alkylene alkylates); hydrophobic polyethers; polyurethanes; polyetheresters; polyacetals; polycyanoacrylates; polyacrylates; polymethylmethacrylates; polysiloxanes; poly(oxyethylene)/poly(oxypropylene) copolymers; polyketals; polyphosphates; polyhydroxyvalerates; polyalkylene oxalates; polyalkylene succinates; poly(maleic acids), as well as copolymers thereof. Biocompatible polymers can also include polyamides, polycarbonates, polyalkylenes, polyalkylene glycols, polyalkylene oxides, polyalkylene terepthalates, polyvinyl alcohols (PVA), methacrylate PVA (m-PVA), polyvinyl ethers, polyvinyl esters, polyvinyl halides, polyvinylpyrrolidone, polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly (methyl methacrylate), poly(ethylmethacrylate), poly(butylmethacrylate), poly(isobutylmethacrylate), poly(hexlmethacrylate), poly(isodecylmethacrylate), poly(lauryl methacrylate), poly (phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), poly(vinyl acetate, poly vinyl chloride polystyrene and polyvinylpryrrolidone, derivatives thereof, linear and branched copolymers and block copolymers thereof, and blends thereof. Exemplary biodegradable polymers include polyesters, poly(ortho esters), poly(ethylene amines), poly(caprolactones), poly(hydroxybutyrates), poly(hydroxyvalerates), polyanhydrides, poly(acrylic acids), polyglycolides, poly(urethanes), polycarbonates, polyphosphate esters, polyphospliazenes, derivatives thereof, linear and branched copolymers and block copolymers thereof, and blends thereof.

59. In some embodiments the particle contains biocompatible and/or biodegradable polyesters or polyanhydrides such as poly(lactic acid), poly(glycolic acid), and poly(lactic-co-glycolic acid). The particles can contain one more of the following polyesters: homopolymers including glycolic acid units, referred to herein as “PGA”, and lactic acid units, such as poly-L-lactic acid, poly-D-lactic acid, poly-D,L-lactic acid, poly-L-lactide, poly-D-lactide, and poly-D,L-lactide5 collectively referred to herein as “PLA”, and caprolactone units, such as poly(e-caprolactone), collectively referred to herein as “PCL”; and copolymers including lactic acid and glycolic acid units, such as various forms of poly(lactic acid-co-glycolic acid) and poly(lactide-co-glycolide) characterized by the ratio of lactic acid:glycolic acid, collectively referred to herein as “PLGA”; and polyacrylates, and derivatives thereof. Exemplary polymers also include copolymers of polyethylene glycol (PEG) and the aforementioned polyesters, such as various forms of PLGA-PEG or PLA-PEG copolymers, collectively referred to herein as “PEGylated polymers”. In certain embodiments, the PEG region can be covalently associated with polymer to yield “PEGylated polymers” by a cleavable linker. In one aspect, the polymer comprises at least 60, 65, 70, 75, 80, 85, 89, 90, 91, 92, 93, 94, 95, 96, 97, 98, or 99 percent acetal pendant groups.

60. The triblock copolymers disclosed herein comprise a core polymer such as, example, polyethylene glycol (PEG), polyvinyl acetate, polyvinyl alcohol, polyvinyl pyrrolidone (PVP), polyethyleneoxide (PEO), poly(vinyl pyrrolidone-co-vinyl acetate), polymethacrylates, polyoxyethylene alkyl ethers, polyoxyethylene castor oils, polycaprolactam, polylactic acid, polyglycolic acid, poly(lactic-glycolic) acid, poly(lactic co-glycolic) acid (PLGA), cellulose derivatives, such as hydroxymethylcellulose, hydroxypropylcellulose and the like.

61. Other particulate systems used to deliver the HSP60 peptides (with or without an additional adjuvant) include carbon nanotubes, silicon dioxide nanoparticles, dendrimers, ferritin nanoparticles, peptide nanocarriers, gold nanoparticles, liposome-polycation-DNA (LPD) complex, oligosaccharide ester derivatives (OEDs) microparticles and combination systems, e.g., liposomes and w/o emulsion.

2. Peptides

a) Protein Variants

62. As discussed herein there are numerous variants of the HSP60 protein and peptides that are known and herein contemplated. In addition, to the known functional HSP60 strain variants there are derivatives of the HSP60 proteins and peptides which also function in the disclosed methods and compositions. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 1
Amino Acid Abbreviations

| Amino Acid | Abbreviations | |
|---|---|---|
| Alanine | Ala | A |
| allosoleucine | AIle | |
| Arginine | Arg | R |
| asparagine | Asn | N |
| aspartic acid | Asp | D |
| Cysteine | Cys | C |
| glutamic acid | Glu | E |
| Glutamine | Gln | Q |
| Glycine | Gly | G |
| Histidine | His | H |
| Isolelucine | Ile | I |
| Leucine | Leu | L |
| Lysine | Lys | K |
| phenylalanine | Phe | F |
| proline | Pro | P |
| pyroglutamic acid | pGlu | |
| Serine | Ser | S |
| Threonine | Thr | T |
| Tyrosine | Tyr | Y |
| Tryptophan | Trp | W |
| Valine | Val | V |

TABLE 2
Amino Acid Substitutions
Original Residue Exemplary Conservative
Substitutions, others are known in the art.

| | |
|---|---|
| Ala | Ser |
| Arg | Lys; Gln |
| Asn | Gln; His |
| Asp | Glu |
| Cys | Ser |
| Gln | Asn, Lys |
| Glu | Asp |
| Gly | Pro |
| His | Asn; Gln |
| Ile | Leu; Val |
| Leu | Ile; Val |
| Lys | Arg; Gln |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

63. Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

64. For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein. For example, a disclosed conservative derivative of SEQ ID NO: 15 is shown in SEQ ID NO: 6, where the isoleucine (I) at positions 2, 3 are changed to a valine (V), the isoleucine (I) at position 13 is changed to a Leucine (L), and the Lysine (K) at residue 7 has been changed to a Glutamine (Q).

65. Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

66. Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

67. It is understood that one way to define the variants and derivatives of the disclosed proteins herein is through defining the variants and derivatives in terms of homology/identity to specific known sequences. For example, SEQ ID NO: 1-13 sets forth a particular sequence of HSP60 peptides and SEQ ID NO: 14 sets forth a particular sequence of a HSP60 protein. Specifically disclosed are variants of these and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

68. Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

69. The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989.

70. It is understood that the description of conservative mutations and homology can be combined together in any combination, such as embodiments that have at least 70% homology to a particular sequence wherein the variants are conservative mutations.

71. As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. It is also understood that while no amino acid sequence indicates what particular DNA sequence encodes that protein within an organism, where particular variants of a disclosed protein are disclosed herein, the known nucleic acid sequence that encodes that protein in the particular HSP60 from which that protein arises is also known and herein disclosed and described.

72. It is understood that there are numerous amino acid and peptide analogs which can be incorporated into the disclosed compositions. For example, there are numerous D amino acids or amino acids which have a different functional substituent then the amino acids shown in Table 1 and Table 2. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way.

73. Molecules can be produced that resemble peptides, but which are not connected via a natural peptide linkage. For example, linkages for amino acids or amino acid analogs can include CH₂NH—, —CH₂S—, —CH₂—CH₂—, —CH═CH— (cis and trans), —COCH₂—, —CH(OH)CH₂—, and —CHH₂SO— (These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CH H₂—S); Hann J. Chem. Soc Perkin Trans. I 307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as b-alanine, g-aminobutyric acid, and the like.

74. Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

75. D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations.

3. Pharmaceutical Carriers/Delivery of Pharmaceutical Products

76. As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

77. The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

78. Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

79. The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

a) Pharmaceutically Acceptable Carriers

80. The compositions, including antibodies, can be used therapeutically in combination with a pharmaceutically acceptable carrier.

81. Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

82. Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

83. Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

84. The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

85. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

86. Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

87. Compositions for oral administration include powders or granules, suspensions or solutions in water or non-aqueous media, capsules, sachets, or tablets. Thickeners, flavorings, diluents, emulsifiers, dispersing aids or binders may be desirable.

88. Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

b) Therapeutic Uses

89. Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

### C. METHOD OF TREATING HEART FAILURE

90. It is understood and herein contemplated that the ability to reduce the inflammatory damage induced by HSP60 can reduce, inhibit, decrease, ameliorate, and/or prevent heart failure, cardiac disease, or cardiac dysfunction and/or any of the symptoms associated therewith (such as, for example, shortness of breath (dyspnea); fatigue and weakness: swelling (edema) of the legs, ankles and feet; rapid or irregular heartbeat; reduced ability to exercise; persistent cough or wheezing with white or pink blood-tinged phlegm; swelling of your abdomen (ascites); and/or chest pain). Thus, disclosed here are methods of treating, reducing, inhibiting, decreasing, ameliorating, and/or preventing heart failure, cardiac disease, or cardiac dysfunction progression to chronic heart failure in a subject comprising administering to the subject any of the HSP60 and adjuvant comprising compositions disclosed herein. For example, in one aspect, disclosed herein are methods of treating, reducing, inhibiting, decreasing, ameliorating, and/or preventing heart failure, cardiac disease, or cardiac dysfunction in a subject comprising administering to the subject a composition comprising an HSP60 peptide (such as, for example, KFGADARALMLQGVD (SEQ ID NO: 1), LMLQGVDLLADAVAV (SEQ ID NO: 2), EKKDRVTDALNATRA (SEQ ID NO: 3), IQSIVPALEIANAHR (SEQ ID NO: 4), AELKKQSKPVT (SEQ ID NO: 5), EVVEGMQFDRGYLSP (SEQ ID NO: 6), KEEKDPGMGAMGGMG (SEQ ID NO: 7), VTDALNATRAAVEEG (SEQ ID NO: 8), TLVLNRLKVGLQVVAVK (SEQ ID NO: 9), EEIAQVATISANG (SEQ ID NO: 10), AVKAPGHFDNRKN (SEQ ID NO: 11), KKQSKPVTTPEE (SEQ ID NO: 12), EIPKEEKDPGMGAMG (SEQ ID NO: 13), or EIIEGMKFDRGYISP (SEQ ID NO: 15)) and an adjuvant (such as, for example, alum, aluminum hydroxide, aluminum phosphate, mixed aluminum salts, a nonionic block co-polymer (such as, for example CRL-1005), Adjuvant system 04 (AS04)(which is a combination of aluminum hydroxide and monophosphoryl lipid A (MPL)), or Adjuvant system 03 (AS03) (which is made up of the oily compounds, D,L-alpha-tocopherol (vitamin E) and squalene, and an emulsifier, polysorbate 80)).

91. Additionally, the ability to reduce the inflammatory damage induced by HSP60 can reduce, inhibit, decrease, ameliorate, and/or prevent the progression of heart failure, cardiac disease, or cardiac dysfunction to chronic heart failure. As noted throughout this application, acute heart failure often leads to death or a progression to chronic heart failure which itself also has no definitive treatment other than heart transplantation. The current 1-year mortality after a single heart failure hospitalization is 36% with an incremental increase with each hospitalization in an individual's lifetime. In one aspect, disclosed here are methods of treating, reducing, inhibiting, decreasing, ameliorating, and/or preventing heart failure, cardiac disease, or cardiac dysfunction progression to chronic heart failure in a subject comprising administering to the subject any of the HSP60 and adjuvant comprising compositions disclosed herein. For example, in one aspect, disclosed herein are methods of treating, reducing, inhibiting, decreasing, ameliorating, and/or preventing heart failure, cardiac disease, or cardiac dysfunction progression to chronic heart failure in a subject comprising administering to the subject a composition comprising an HSP60 peptide (such as, for example, KFGADARALMLQGVD (SEQ ID NO: 1), LMLQGVDLLADAVAV (SEQ ID NO: 2), EKKDRVTDALNATRA (SEQ ID NO: 3), IQSIVPALEIANAHR (SEQ ID NO: 4), AELKKQSKPVT (SEQ ID NO: 5), EVVEGMQFDRGYLSP (SEQ ID NO: 6), KEEKDPGMGAMGGMG (SEQ ID NO: 7), VTDALNATRAAVEEG (SEQ ID NO: 8), TLVLNRLKVGLQVVAVK (SEQ ID NO: 9), EEIAQVATISANG (SEQ ID NO: 10), AVKAPGHFDNRKN (SEQ ID NO: 11), KKQSKPVTTPEE (SEQ ID NO: 12), EIPKEEKDPGMGAMG (SEQ ID NO: 13), or EIIEGMKFDRGYISP (SEQ ID NO: 15)) and an adjuvant (such as, for example, alum, aluminum hydroxide, aluminum phosphate, mixed aluminum salts, a nonionic block co-polymer (such as, for example CRL-1005), Adjuvant system 04 (AS04)(which is a combination of aluminum hydroxide and monophosphoryl lipid A (MPL)), or Adjuvant system 03 (AS03) (which is made up of the oily compounds, D,L-alpha-tocopherol (vitamin E) and squalene, and an emulsifier, polysorbate 80)).

92. In some instances, the heart failure that can be treated using the disclosed methods can be the result of increased damage from a cardiac disease or dysfunction. It is understood and herein contemplated that HSP60 peptide and adjuvant comprising compositions disclosed herein can be used in treating, reducing, inhibiting, decreasing, ameliorating, and/or preventing the progression of a cardiac disease or cardiac dysfunction to heart failure and thus decrease the risk of heart failure, decrease the severity of heart failure, and/or otherwise inhibit or treat heart failure. Accordingly, disclosed herein are methods of treating, reducing, inhibiting, decreasing, ameliorating, and/or preventing heart failure, cardiac disease, dysfunction, as well as, methods of treating, reducing, inhibiting, decreasing, ameliorating, and/or preventing heart failure, cardiac disease, dysfunction progression to chronic heart failure; wherein the cardiac disease or cardiac dysfunction comprises coronary artery disease, myocardial infarction, hypertension, cardiomyopathy, myocarditis, congenital heart defect, ischemia reperfusion injury, myocardial ischemia, myocardial reperfusion, subendocardial ischemia, Takayasu's arteritis, atrial fibrillation, hemorrhagic strokes, transient ischemia attack, or heart arrythmias. Additionally, diabetes, HIV, hyperthyroidism, hypothyroidism, or a buildup of iron (hemochromatosis) or protein (amyloidosis), viruses that attack the heart muscle, severe infections, allergic reactions, blood clots in the lungs, the use of certain medications, or any illness that affects the whole body can contribute to heart failure.

93. It is also understood that heart failure treated by the disclosed methods does not have to be the result of cardiac damage from disease or dysfunction, but can also be the result of cardiac damage as the result of a medical treatment. For example, the heart failure is surgically induced (such as, for example, surgically induced ischemic/reperfusion events occurring during the preservation of organs for transplant or during cardiac surgery (including coronary artery bypass surgery, stent implantation, heart valve replacements, myectomy, transmyocardial revascularization, congenital heart surgery, angioplasty, atherectomy, and/or cardiomyoplasty. Thus, in one aspect, disclosed herein are methods of treating, reducing, inhibiting, decreasing, ameliorating, and/or preventing heart failure, cardiac disease, dysfunction, as well as, methods of treating, reducing, inhibiting, decreasing, ameliorating, and/or preventing heart failure, cardiac disease, or cardiac dysfunction progression to chronic heart failure; wherein the heart failure is surgically induced (such as, for example, surgically induced ischemic/reperfusion events occurring during the preservation of organs for transplant or during cardiac surgery (including coronary artery bypass surgery, stent implantation, heart valve replacements, myectomy, transmyocardial revascularization, congenital heart surgery, angioplasty, atherectomy, and/or cardiomyoplasty).

94. The disclosed methods are designed in one aspect to be a prophylaxis to heart disease ultimately inhibiting, reducing, or preventing the occurrence, severity, or delaying the onset of heart failure, progression to chronic heart failure, or further damage to the heart. In such instances, the compositions disclosed herein comprising an HSP60 peptide and an adjuvant can be administered after the presence of a cardiac disease or dysfunction, but prior to the onset of heart failure, or complete heart failure occurring. Similarly, where there is a risk of heart failure or increased damage that can lead to heart failure due to a medical procedure the compositions comprising a HSP60 peptide and an adjuvant disclosed herein can be administered before any surgical procedure, during, surgery, or after surgery, but prior to any heart failure or symptoms thereof. In one aspect, the compositions comprising a HSP60 peptide and an adjuvant can be administered to a subject as risk for heart failure, undergoing a medical procedure that has increased risk for heart failure (such as, for example, surgically induced ischemic/reperfusion events occurring during the preservation of organs for transplant or during cardiac surgery (including coronary artery bypass surgery, stent implantation, heart valve replacements, myectomy, transmyocardial revascularization, congenital heart surgery, angioplasty, atherectomy, and/or cardiomyoplasty), wherein the compositions comprising a HSP60 peptide and an adjuvant is administered at the time of surgery or at least 1, 2, 3, 4, 5, 6 7, 8, 9, 10, 11, 12, 13, 14, 15, 16, 17, 18, 19, 20, 21, 22, 23, 24, 26, 28, 30, 32, 34, 35, 36, 38, 40, 42, 44, 46, 48, 54, 60, 66, 72, 78, 84, 90, 96 hours, 5, 6, 7, 8, 9, 10, 11, 12, 13, 14, 15, 16, 17, 18, 19, 20, 21, 22, 23, 24, 25, 26, 27, 28, 29, 30, 31, 35, 42, 49, 54, 60, 63, 70, 77, 84, 90, 93 days, 4, 5, 6, 7, 8, 9, 10, 11, or 12 months prior to undergoing a medical procedure that increases the risk of heart failure or increases the risk of cardiac damage that can lead to heart failure.

95. In one aspect, it is understood and herein contemplated that heart failure can be acute or chronic. Thus, not all treatment has to be prophylactic, but the disclosed methods can also be used therapeutically, after the onset of heart failure or after one or more symptoms of heart failure has been detected. Accordingly, disclosed herein are methods of treating, reducing, inhibiting, decreasing, ameliorating, and/or preventing heart failure, cardiac disease, dysfunction and/or methods of treating, reducing, inhibiting, decreasing, ameliorating, and/or preventing heart failure, cardiac disease, or cardiac dysfunction progression to chronic heart failure, wherein the heart failure is a chronic heart failure and the composition is administered to the subject after the onset of heart failure, or wherein the heart failure is acute heart failure and the composition is administered to the subject after the onset of symptoms associated with heart failure. In some instances, wherein the heart failure is surgically induced, the composition comprising an HSP60 peptide and an adjuvant is administered at least 1, 2, 3, 4, 5, 6 7, 8, 9, 10, 11, 12, 13, 14, 15, 16, 17, 18, 19, 20, 21, 22, 23, 24, 26, 28, 30, 32, 34, 35, 36, 38, 40, 42, 44, 46, 48, 54, 60, 66, 72, 78, 84, 90, 96 hours, 5, 6, 7, 8, 9, 10, 11, 12, 13, 14, 15, 16, 17, 18, 19, 20, 21, 22, 23, 24, 25, 26, 27, 28, 29, 30, 31, 35, 42, 49, 54, 60, 63, 70, 77, 84, 90, 93 days, 4, 5, 6, 7, 8, 9, 10, 11, or 12 months after surgery.

96. While current therapies focus on the neurohormonal system in chronic heart failure, they still only demonstrate a slowing of the progression and do not address the initial onset and underlying processes which occur in the acute setting. The underlying mechanism which contributes significantly to the worsening prognosis is apoptosis or programmed cell death.

While such apoptosis occurs in all forms of heart failure and is responsible for the loss of contractile cells, it additionally can activate the neurohormonal system and prove to be one of the underlying causes that ultimately leads to chronic heart failure and death. Blocking this activation early can have long term effects and stop or delay the onset of chronic heart failure. Accordingly, in some aspects, disclosed herein are methods of treating, reducing, inhibiting, decreasing, ameliorating, and/or preventing apoptosis associated with heart failure, cardiac disease, or cardiac dysfunction in a subject comprising administering to the subject a composition comprising an HSP60 peptide (such as, for example, KFGADARALMLQGVD (SEQ ID NO: 1), LMLQGVDLLADAVAV (SEQ ID NO: 2), EKKDRVTDALNATRA (SEQ ID NO: 3), IQSIVPALEIANAHR (SEQ ID NO: 4), AELKKQSKPVT (SEQ ID NO: 5), EVVEGMQFDRGYLSP (SEQ ID NO: 6), KEEKDPGMGAMGGMG (SEQ ID NO: 7), VTDALNATRAAVEEG (SEQ ID NO: 8), TLVLNRLKVGLQVVAVK (SEQ ID NO: 9), EEIAQVATISANG (SEQ ID NO: 10), AVKAPGHFDNRKN (SEQ ID NO: 11), KKQSKPVTTPEE (SEQ ID NO: 12), EIPKEEKDPGMGAMG (SEQ ID NO: 13), or EIIEGMKFDRGYISP (SEQ ID NO: 15)) and an adjuvant (such as, for example, alum, aluminum hydroxide, aluminum phosphate, mixed aluminum salts, a nonionic block co-polymer (such as, for example CRL-1005), Adjuvant system 04 (AS04)(which is a combination of aluminum hydroxide and monophosphoryl lipid A (MPL)), or Adjuvant system 03 (AS03) (which is made up of the oily compounds, D,L-alpha-tocopherol (vitamin E) and squalene, and an emulsifier, polysorbate 80)).

### D. EXAMPLES

97. The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

98. Using human tissues from explanted hearts at the time of transplantation and a mouse model of heart failure, several key proteins were identified that are intimately involved in heart failure. The vaccine disclosed herein is focused on one of these molecules, HSP60, which is key in how heart failure continues to worsen. The currently proposed therapy is therefore different than all other approved therapies as it is focused on the blocking of this molecule by using the native immune system to absorb the HSP60 molecule before it can induce further damage to the heart.

99. The initial tests were performed on a mouse model of heart failure which is induced by an increase in blood pressure. This induction is similar to non-ischemic heart failure in humans and represents approximately 50% of all heart failure cases. Initially, the whole molecule of HSP60 which was compounded with an Alum molecule was injected. This compounded molecule of HSP60 and Alum was given to normal mice to determine the best delivery method to induce maximal immune responses by identifying the presence of IgG specific antibodies to the HSP60 whole molecule. This delivery method was then used to “vaccinate” mice before the initiation of the heart failure protocol used in mouse animal model. The animals were then imaged by Magnetic Resonance Imaging (MRI) to determine any effect on the contractile properties of the heart with the vaccine (Heart Failure+Alum-HSP60) in comparison to normal mice (Control) and mice which had undergone only heart failure (Heart Failure) induction without the vaccine. As shown in FIG. 1, the ejection fraction of the heart decreased in heart failure but remained higher in those animals treated with the therapy. Additional indicators such as End Diastolic Diameter and LV Mass were consistant with this observed improvement in function. This shows that there was a significant preservation of function in those animals which were given the vaccine.

100. Following the MRI data the heart was investigated for other markers that are well known in heart failure including fibrosis which is a histological hallmark of heart failure. The fibrosis is not only diagnostic but adds additional strain on the heart by increasing stiffness and decrease the ability of the heart to both contract and relax during a cardiac cycle. FIG. 2 shows the fibrosis staining (blue color) in two representative animals in each group. Quantitation of the fibrosis is shown in FIG. 3 and clearly demonstrates an increase in fibrosis in heart failure which is greatly reduced in the vaccine treated animals. This demonstrates the inhibition of fibrosis normally associated with heart failure by the vaccine.

101. The heart also increases in size as it progresses to heart failure which further decreases its ability to contract and pump sufficient blood to maintain proper perfusion of the organs. In the mouse model of heart failure there is a very dramatic increase in heart weight as shown in FIG. 4 and this increase in weight is partially blocked by the use of the Alum-HSP60 vaccine.

102. Additional experiments have been performed to determine if apoptosis, which is known to be high in heart failure, is decreased in response to the vaccine treatment. As shown in the following FIG. 4, apoptosis is decreased in the vaccine group (Heart Failure+Alum-HSP60).

103. Additionally, peptides derived from the HSP60 sequence can be used in combination with Alum can provide a more powerful and more specific vaccine than the use of the whole protein which was used in the preliminary data above. Immunization with the whole HSP60 protein is effective in the animal model of heart failure. A vaccine however is more likely to be more specific and more efficacious if the antigen presented is a smaller subset of the full-length protein. A peptide response can decrease side effects while simultaneously increasing the effectiveness of a vaccine. With that in mind, peptide sequence regions were designed which were the most likely to produce a response and can be used preferentially over the whole protein. To accomplish this intelligent design techniques was used to predict which peptides would be most antigenic using tools available from the National Institute of Allergy and Infectious Diseases (Antibody Epitope Prediction), US National Library of Medicine (Blast sequence alignment) and protein folding prediction databases.

### E. SEQUENCES

SEQ ID NO: 1
KFGADARALMLQGVD
SEQ ID NO: 2
LMLQGVDLLADAVAV
SEQ ID NO: 3
EKKDRVTDALNATRA
SEQ ID NO: 4
IQSIVPALEIANAHR
SEQ ID NO: 5
AELKKQSKPVT
SEQ ID NO: 6
EVVEGMQFDRGYLSP
SEQ ID NO: 7
KEEKDPGMGAMGGMG
SEQ ID NO: 8
VTDALNATRAAVEEG
SEQ ID NO: 9
TLVLNRLKVGLQVVAVK
SEQ ID NO: 10
EEIAQVATISANG
SEQ ID NO: 11
AVKAPGHFDNRKN
SEQ ID NO: 12
KKQSKPVTTPEE
SEQ ID NO: 13
EIPKEEKDPGMGAMG
SEQ ID NO: 14: amino acid sequence for human HSP60

| | |
|---|---|
| 1 | mlrlptvfrq mrpvsrvlap hltrayakdv kfgadaralm |
| | lqgvdllada vavtmgpkgr |
| 61 | tviieqswgs pkvtkdgvtv aksidikdky knigaklvqd |
| | vanntneeag dgtttatvla |
| 121 | rsiakegfek iskganpvei rrgvmlavda viaelkkqsk |
| | pvttpeeiaq vatisangdk |
| 181 | eigniisdam kkvgrkgvit vkdgktlnde leiiegmkfd |
| | rgyispyfin tskgqkcefq |
| 241 | dayvllsekk issiqsivpa leianahrkp lviiaedvdg |
| | ealstlvlnr lkvglqvvav |
| 301 | kapgfgdnrk nqlkdmaiat ggavfgeegl tlnledvqph |
| | dlgkvgeviv tkddamllkg |
| 361 | kgdkaqiekr iqeiieqldv ttseyekekl nerlaklsdg |
| | vavlkvggts dvevnekkdr |
| 421 | vtdalnatra aveegivlgg gcallrcipa ldsltpaned |
| | qkigieiikr tlkipamtia |
| 481 | knagvegsli vekimqssse vgydamagdf vnmvekgiid |
| | ptkvvrtall daagvasllt |
| 541 | taevvvteip keekdpgmga mggmgggmgg gmf |

SEQ ID NO: 15
EIIEGMKFDRGYISP

1. A composition comprising a HSP60 peptide and an adjuvant.

2. The composition of claim 1, wherein the HSP60 peptide comprises KFGADARALMLQGVD (SEQ ID NO: 1), LMLQGVDLLADAVAV (SEQ ID NO: 2), EKKDRVTDALNATRA (SEQ ID NO: 3), IQSIVPALEIANAHR (SEQ ID NO: 4), AELKKQSKPVT (SEQ ID NO: 5), EVVEGMQFDRGYLSP (SEQ ID NO: 6), KEEKDPGMGAMGGMG (SEQ ID NO: 7), VTDALNATRAAVEEG (SEQ ID NO: 8), TLVLNRLKVGLQVVAVK (SEQ ID NO: 9), EEIAQVATISANG (SEQ ID NO: 10), AVKAPGHFDNRKN (SEQ ID NO: 11), KKQSKPVTTPEE (SEQ ID NO: 12), EIPKEEKDPGMGAMG (SEQ ID NO: 13), or EIIEGMKFDRGYISP (SEQ ID NO: 15).

3. The composition of claim 1, wherein the peptide is conjugated to the adjuvant.

4. The composition of claim 1, wherein the adjuvant comprises alum, aluminum hydroxide, aluminum phosphate, mixed aluminum salts, a nonionic block co-polymer, Adjuvant system 04 (AS04), or Adjuvant system 03 (AS03).

5. A method of treating, reducing, inhibiting, decreasing, ameliorating, and/or preventing heart failure, cardiac disease, cardiac dysfunction, or the progression of heart failure, cardiac disease or cardiac dysfunction to chronic heart failure in a subject comprising administering to the subject the composition of claim 1.

6. A method of treating, reducing, inhibiting, decreasing, ameliorating, and/or preventing heart failure, cardiac disease, or cardiac dysfunction progression to chronic heart failure in a subject comprising administering to the subject a composition comprising a HSP60 peptide and an adjuvant.

7. The method of treating, reducing, inhibiting, decreasing, ameliorating, and/or preventing heart failure, cardiac disease, or cardiac dysfunction progression to chronic heart failure of claim 6, wherein the HSP60 peptide comprises KFGADARALMLQGVD (SEQ ID NO: 1), LMLQGVDLLADAVAV (SEQ ID NO: 2), EKKDRVTDALNATRA (SEQ ID NO: 3), IQSIVPALEIANAHR (SEQ ID NO: 4), AELKKQSKPVT (SEQ ID NO: 5), EVVEGMQFDRGYLSP (SEQ ID NO: 6), KEEKDPGMGAMGGMG (SEQ ID NO: 7), VTDALNATRAAVEEG (SEQ ID NO: 8), TLVLNRLKVGLQVVAVK (SEQ ID NO: 9), EEIAQVATISANG (SEQ ID NO: 10), AVKAPGHFDNRKN (SEQ ID NO: 11), KKQSKPVTTPEE (SEQ ID NO: 12), EIPKEEKDPGMGAMG (SEQ ID NO: 13), or EIIEGMKFDRGYISP (SEQ ID NO: 15).

8. The method of treating, reducing, inhibiting, decreasing, ameliorating, and/or preventing heart failure, cardiac disease, or cardiac dysfunction progression to chronic heart failure of claim 6, wherein the adjuvant comprises Alum.

9. The method of treating, reducing, inhibiting, decreasing, ameliorating, and/or preventing heart failure, cardiac disease, or cardiac dysfunction progression to chronic heart failure of claim 6, wherein the cardiac disease or cardiac dysfunction comprises coronary artery disease, myocardial infarction, hypertension, cardiomyopathy, myocarditis, congenital heart defect, ischemia reperfusion injury, myocardial ischemia, myocardial reperfusion, subendocardial ischemia, Takayasu's arteritis, atrial fibrillation, hemorrhagic strokes, transient ischemia attack, or heart arrythmias.

10. The method of treating, reducing, inhibiting, decreasing, ameliorating, and/or preventing heart failure, cardiac disease, or cardiac dysfunction progression to chronic heart failure of claim 6, wherein the heart failure is acute.

11. The method of treating, reducing, inhibiting, decreasing, ameliorating, and/or preventing heart failure, cardiac disease, or cardiac dysfunction progression to chronic heart failure of claim 6, wherein the heart failure is surgically induced.

12. The method of treating, reducing, inhibiting, decreasing, ameliorating, and/or preventing heart failure, cardiac disease, or cardiac dysfunction progression to chronic heart failure of claim 6, wherein the composition is administered to the subject prior to the onset of heart failure.

13. The method of treating, reducing, inhibiting, decreasing, ameliorating, and/or preventing heart failure, cardiac disease, or cardiac dysfunction progression to chronic heart failure of claim 6, wherein the heart failure is acute heart failure and the composition is administered to the subject after the onset of symptoms associated with heart failure.

14. A method of treating, reducing, inhibiting, decreasing, ameliorating, and/or preventing heart failure, cardiac disease, or cardiac dysfunction in a subject comprising administering to the subject a composition comprising a HSP60 peptide and an adjuvant.

15. The method of treating, reducing, inhibiting, decreasing, ameliorating, and/or preventing heart failure, cardiac disease, or cardiac dysfunction of claim 14, wherein the HSP60 peptide comprises KFGADARALMLQGVD (SEQ ID NO: 1), LMLQGVDLLADAVAV (SEQ ID NO: 2), EKKDRVTDALNATRA (SEQ ID NO: 3), IQSIVPALEIANAHR (SEQ ID NO: 4), AELKKQSKPVT (SEQ ID NO: 5), EVVEGMQFDRGYLSP (SEQ ID NO: 6), KEEKDPGMGAMGGMG (SEQ ID NO: 7), VTDALNATRAAVEEG (SEQ ID NO: 8), TLVLNRLKVGLQVVAVK (SEQ ID NO: 9), EEIAQVATISANG (SEQ ID NO: 10), AVKAPGHFDNRKN (SEQ ID NO: 11), KKQSKPVTTPEE (SEQ ID NO: 12), EIPKEEKDPGMGAMG (SEQ ID NO: 13), or EIIEGMKFDRGYISP (SEQ ID NO: 15).

16. The method of treating, reducing, inhibiting, decreasing, ameliorating, and/or preventing heart failure, cardiac disease, or cardiac dysfunction of claim 14, wherein the adjuvant comprises Alum.

17. The method of treating, reducing, inhibiting, decreasing, ameliorating, and/or preventing heart failure, cardiac disease, or cardiac dysfunction of claim 14, wherein the cardiac disease or dysfunction comprises coronary artery disease, myocardial infarction, hypertension, cardiomyopathy, myocarditis, congenital heart defect, ischemia reperfusion injury, myocardial ischemia, myocardial reperfusion, subendocardial ischemia, Takayasu's arteritis, atrial fibrillation, hemorrhagic strokes, transient ischemia attack, or heart arrythmias.

18. The method of treating, reducing, inhibiting, decreasing, ameliorating, and/or preventing heart failure, cardiac disease, or cardiac dysfunction of claim 14, wherein the heart failure is acute.

19. The method of treating, reducing, inhibiting, decreasing, ameliorating, and/or preventing heart failure, cardiac disease, or cardiac dysfunction of claim 14, wherein the heart failure is surgically induced.

20. The method of treating, reducing, inhibiting, decreasing, ameliorating, and/or preventing heart failure, cardiac disease, or cardiac dysfunction of claim 14, wherein the composition is administered to the subject prior to the onset of heart failure.

21. The method of treating, reducing, inhibiting, decreasing, ameliorating, and/or preventing heart failure, cardiac disease, or cardiac dysfunction of claim 14, wherein the heart failure is a chronic heart failure and the composition is administered to the subject after the onset of heart failure.

22. The method of treating, reducing, inhibiting, decreasing, ameliorating, and/or preventing heart failure, cardiac disease, or cardiac dysfunction of claim 14, wherein the heart failure is acute heart failure and the composition is administered to the subject after the onset of symptoms associated with heart failure.
